(19) United States
(12) Patent Application Publication
(10) Pub. No.: US 2018/0297385 A1
(43) Pub. Date: Oct. 18, 2018

(54) APPARATUS, METHOD, AND STORAGE MEDIUM FOR DETERMINING RECORDING AMOUNT OF RECORDING MATERIAL

(71) Applicant: CANON KABUSHIKI KAISHA, Tokyo (JP)

(72) Inventors: Takahiro Suzuki, Tokyo (JP); Chika Inoshita, Kawasaki-shi (JP)

(21) Appl. No.: 15/945,504

(22) Filed: Apr. 4, 2018

(30) Foreign Application Priority Data
Apr. 12, 2017 (JP) 2017-079152
Dec. 20, 2017 (JP) 2017-243381

(51) Int. Cl. B41M 5/00; B41J 2/21; G01N 33/32

(52) U.S. Cl. CPC B41M 5/0088; G01N 33/32; B41J 2/2132

(57) ABSTRACT

An apparatus configured to determine a first recording amount of a colored recording material to be recorded on a protruded portion and a second recording amount of a colored recording material to be recorded on a depressed portion includes a first acquisition unit configured to acquire color information specifying a first color and a second color, a calculation unit configured to calculate based on the color information a color combination of a color to be recorded on the protruded portion and a color to be recorded on the depressed portion, a second acquisition unit configured to acquire based on the color combination a recording amount combination of the first recording amount and the second recording amount, and a determination unit configured to select one combination according to a predetermined condition from the recording amount combination.

### BACKGROUND OF THE INVENTION

### Field of the Invention

The aspect of the embodiments relates to an image processing technique for reproducing colors on a recording medium with protrusions and depressions formed thereon.

### Description of the Related Art

In recent years, printers (hereinafter, “UV printers”) which employ ultraviolet (UV) curable resin inks (hereinafter, “UV inks”) are commercially available. The UV printers irradiate the UV inks with UV rays to cure the UV inks. The UV printers repeatedly apply and cure the UV inks to form protrusions and depressions on the surface of a printed matter. The protrusions and depressions on the surface of the printed matter affect the reflection characteristics of incident light such as a reflection direction and reflection intensity. Thus, the reflection characteristics of a printed matter are controllable by controlling the protrusions and depressions on the surface of the printed matter in addition to conventional color control.

A technique for controlling the reflection characteristics of printed matters by controlling protrusions and depressions and colors is discussed in Japanese Patent Application Laid-Open No. 2017-052154. Japanese Patent Application Laid-Open No. 2017-052154 discusses a technique in which fine protrusions and depressions in the shape of parallel lines are formed and different colors are applied to a protruded portion and a depressed portion of the protrusions and depressions. When the protruded and depressed portions of the printed matter formed by the technique discussed in Japanese Patent Application Laid-Open No. 2017-052154 are observed by an observer, the area ratio of the protruded and depressed portions that are visually recognized by the observer differs depending on the observation angle. Thus, when the printed matter is observed, the colors of the printed matter are observed differently depending on the observation angle.

However, a recording material recorded on a recording medium is likely to spread after being applied onto the recording medium, so the recording material on the protruded portion of the protrusions and depressions is likely to flow into the depressed portion. This creates a situation that the colors intended to be reproduced by applying different recording materials onto the protruded portion and the depressed portion cannot be reproduced with great accuracy.

### SUMMARY OF THE INVENTION

According to an aspect of the embodiments, an apparatus configured to determine a first recording amount of a colored recording material to be recorded on a protruded portion and a second recording amount of a colored recording material to be recorded on a depressed portion to form an image by recording the plurality of types of colored recording materials on the protrusion and the depression on the recording medium so that different colors are visually recognized from the image when the image is observed from at least two different directions of azimuth angles includes a first acquisition unit configured to acquire color information specifying a first color and a second color, a calculation unit configured to calculate, based on the color information, a first color combination of a color to be recorded on the protruded portion and a color to be recorded on the depressed portion for a case in which the first color is visually recognized when the image is observed from a first direction and the second color is visually recognized when the image is observed from a second direction, and a second color combination of the color to be recorded on the protruded portion and the color to be recorded on the depressed portion for a case in which the second color is visually recognized when the image is observed from the first direction and the first color is visually recognized when the image is observed from the second direction, a second acquisition unit configured to acquire a first recording amount combination of the first recording amount and the second recording amount based on the first color combination and acquire a second recording amount combination of the first recording amount and the second recording amount based on the second color combination, and a determination unit configured to select one combination according to a predetermined condition from the first recording amount combination and the second recording amount combination.

Further features of the disclosure will become apparent from the following description of exemplary embodiments with reference to the attached drawings.

### BRIEF DESCRIPTION OF THE DRAWINGS

FIG. 1 is a block diagram illustrating a hardware configuration of an image processing apparatus.

FIG. 2 is a block diagram illustrating a logical configuration of the image processing apparatus.

FIG. 3 is a flowchart illustrating a process which is executed by the image processing apparatus.

FIGS. 4A, 4B, and 4C are diagrams illustrating an example of a user interface (UI) screen.

FIGS. 5A and 5B are diagrams illustrating an example of the protrusions and depressions (uneven shape) for reproducing anisotropy.

FIG. 6 is a diagram illustrating an example of a combination of recording amounts of colored inks.

FIG. 7 is a flowchart illustrating a process of determining the combination of recording amounts of colored inks.

FIG. 8 is a diagram illustrating an example of a color conversion table.

FIG. 9 is a flowchart illustrating a process of generating formation data.

FIG. 10 is a flowchart illustrating a process of determining a combination of recording amounts of colored inks.

FIG. 11 is a diagram illustrating how a printed matter is observed from different elevation angle directions.

FIGS. 12A and 12B are schematic diagrams illustrating how an image of a reproduction target object is captured under two different geometric conditions.

FIG. 13 is a diagram illustrating an example of a color conversion table.

FIG. 14 is a flowchart illustrating a process of determining a combination of recording amounts of colored inks.

FIG. 15 is a flowchart illustrating a process of determining a combination of recording amounts of colored inks.

### DESCRIPTION OF THE EMBODIMENTS

### <Hardware Configuration of Image Processing Apparatus 1>

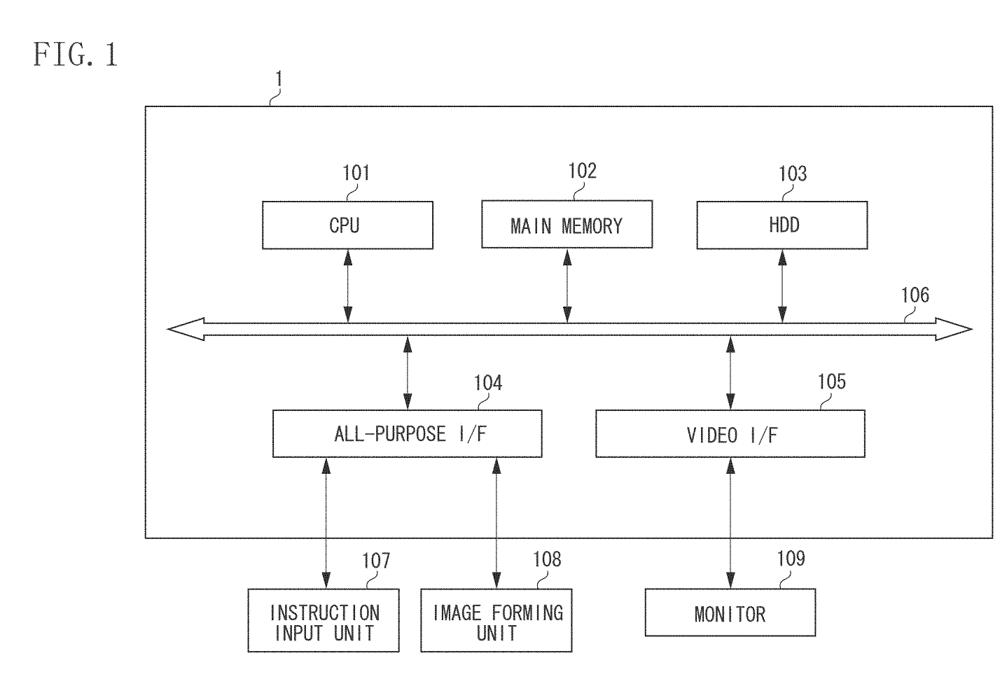

An image processing apparatus 1 according to a first exemplary embodiment will be described below. FIG. 1 is a block diagram illustrating a hardware configuration of the image processing apparatus 1. The image processing apparatus 1 includes a central processing unit (CPU) 101, a main memory 102, a hard disk drive (HDD) 103, an all-purpose interface (I/F) 104, a video I/F 105, and a main bus 106. An instruction input unit 107, such as a keyboard and a mouse, and an image forming unit 108 are connected to the main bus 106 via the all-purpose I/F 104. A monitor 109 is connected to the main bus 106 via the video I/F 105. While the image processing apparatus 1 is described as a personal computer (PC), the image processing apparatus 1 is not limited to the PCs and can be a microcomputer, etc.

The CPU 101 operates various types of software (computer programs) stored in the HDD 103 to execute various types of processing described below. First, the CPU 101 activates an image processing application stored in the HDD 103 according to a user instruction given via the instruction input unit 107. Next, the CPU 101 loads the image processing application onto the main memory 102 and displays a user interface (UI) screen on the monitor 109. Then, various types of data stored in the HDD 103 are transferred to the main memory 102 via the main bus 106 based on an instruction from the CPU 101. The data transferred to the main memory 102 undergoes predetermined computation processing according to an instruction from the CPU 101. A result of the computation processing is transmitted to the monitor 109 or the image forming unit 108 via the main bus 106. Alternatively, the result (data) obtained by the computation processing can be stored in the HDD 103 or an external storage device instead of being transmitted to the image forming unit 108 or the monitor 109.

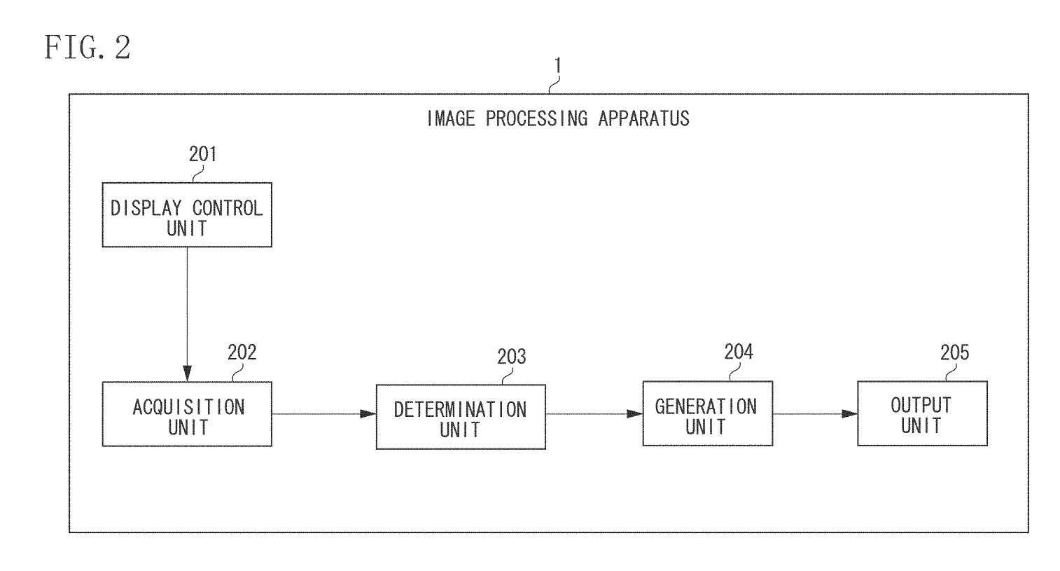

The following describes a process which is executed by the image processing application based on an instruction from the CPU 101 in the hardware configuration described above. FIG. 2 is a block diagram illustrating a logical configuration of the image processing apparatus 1 in the present exemplary embodiment. The image processing apparatus 1 includes a display control unit 201, an acquisition unit 202, a determination unit 203, a generation unit 204, and an output unit 205. The display control unit 201 displays on the monitor 109 the UI screen for receiving user instruction input. The acquisition unit 202 acquires two pieces of color information based on a user instruction input via the UI screen. The determination unit 203 determines based on the two pieces of acquired color information a combination of recording amounts of colored inks to be recorded on a protruded portion to be formed on a recording medium and a combination of recording amounts of colored inks to be recorded on a depressed portion to be formed on the recording medium. The generation unit 204 generates formation data for forming protrusions and depressions on the recording medium and recording colored inks on the protrusions and depressions based on the determined combinations of recording amounts. The output unit 205 outputs the generated formation data to the image forming unit 108, the monitor 109, etc. The image forming unit 108 in the present exemplary embodiment is a UV printer which employs UV inks and is capable of forming protrusions and depressions on the surface of a printed matter by repeatedly applying and curing the UV inks. While the UV printer is described as an inkjet printer in the present exemplary embodiment, the UV printer can be a printer of a different method such as an electrophotographic printer.

### <Printed Matter Formed by Image Forming Unit 108>

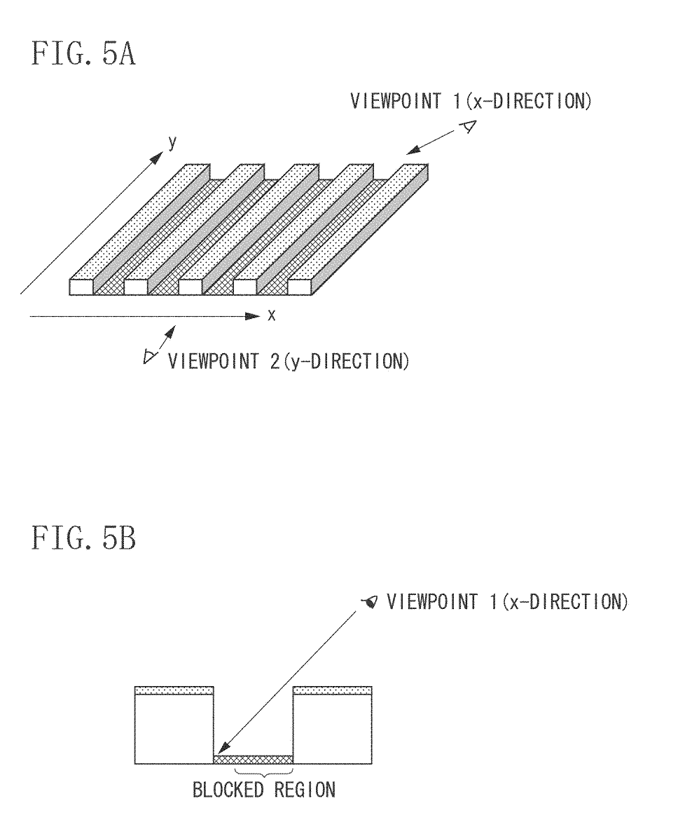

A printed matter formed by the image forming unit 108 has a surface with protrusions and depressions (an uneven shape) with a pattern of parallel lines as illustrated in FIG. 5A. The uneven shape has a protruded portion and a depressed portion. The protruded portion is a region formed by curing the UV inks on the recording medium. The depressed portion is a region where no protruded portion is formed. FIG. 5B illustrates a region blocked by the protruded portion when the printed matter illustrated in FIG. 5A is observed from an x-direction (viewpoint 1). As illustrated in FIG. 5B, when the printed matter is observed from the viewpoint 1 (x-direction), the depressed portion is blocked by the protruded portion. Thus, in the case where the colored inks are recorded on the protruded and depressed portions, only the color of the colored ink recorded on the protruded portion is observed by the observer. On the other hand, when the printed matter is observed from a viewpoint 2 (y-direction), the depressed portion is not blocked by the protruded portion. Thus, in the case where the colored inks are recorded on the protruded and depressed portions, a mixed color of the color of the colored ink recorded on the protruded portion and the color of the colored ink recorded on the depressed portion is observed by the observer. As described above, the colored inks exhibiting different colors are respectively applied to the protruded portion and the depressed portion to form a print (hereinafter, “anisotropic print”) from which different colors are visually recognized from two directions of different azimuth angles. The viewpoint 1 is the viewpoint in the case of observing the printed matter from the direction which is orthogonal to the protruded portion of the pattern of parallel lines, whereas the viewpoint 2 is the viewpoint in the case of observing the printed matter from the direction which is parallel to the protruded portion of the pattern of parallel lines. Further, the difference in azimuth angle between the viewpoints 1 and 2 is 90 degrees. Further, the elevation angle of the viewpoint 1 is the angle at which the depressed portion is blocked by the protruded portion, and the elevation angle of the viewpoint 2 is the same angle as the elevation angle of the viewpoint 1. In the present exemplary embodiment, anisotropy is a characteristic that different colors are visually recognized from two directions of different azimuth angles.

### <Flow of Process Executed by Image Processing Apparatus 1>

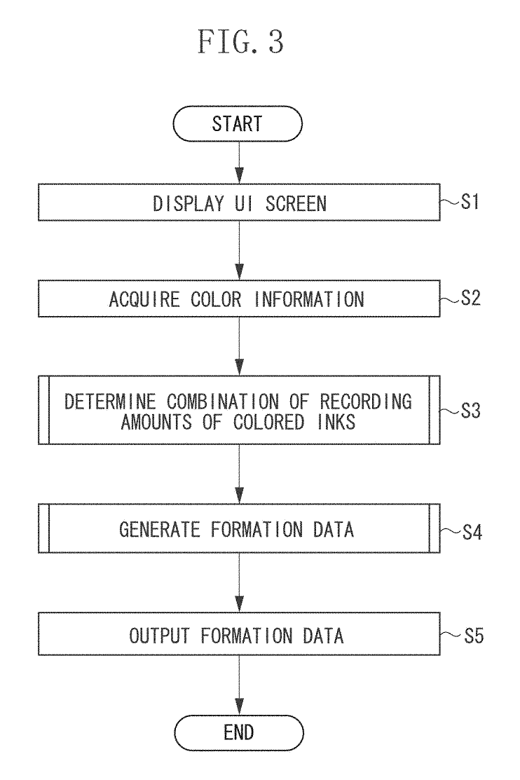

FIG. 3 is a flowchart illustrating a process executed by the image processing apparatus 1. Details of operations executed in the image processing apparatus 1 will be described below with reference to FIG. 3.

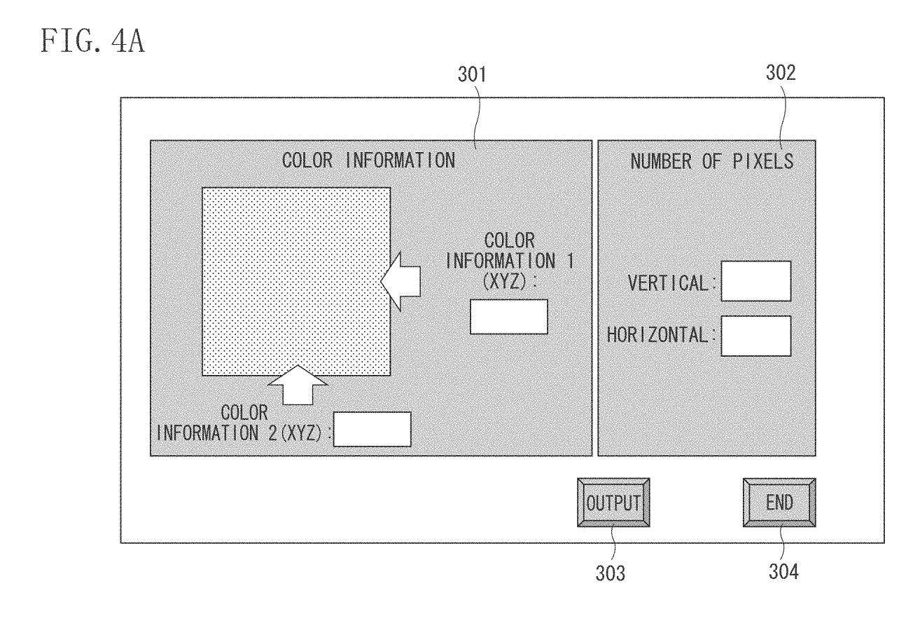

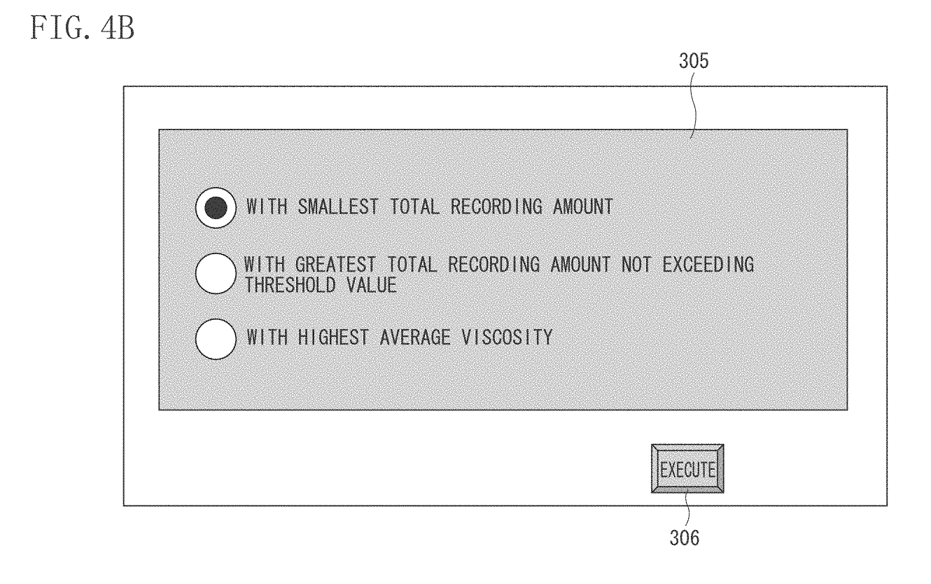

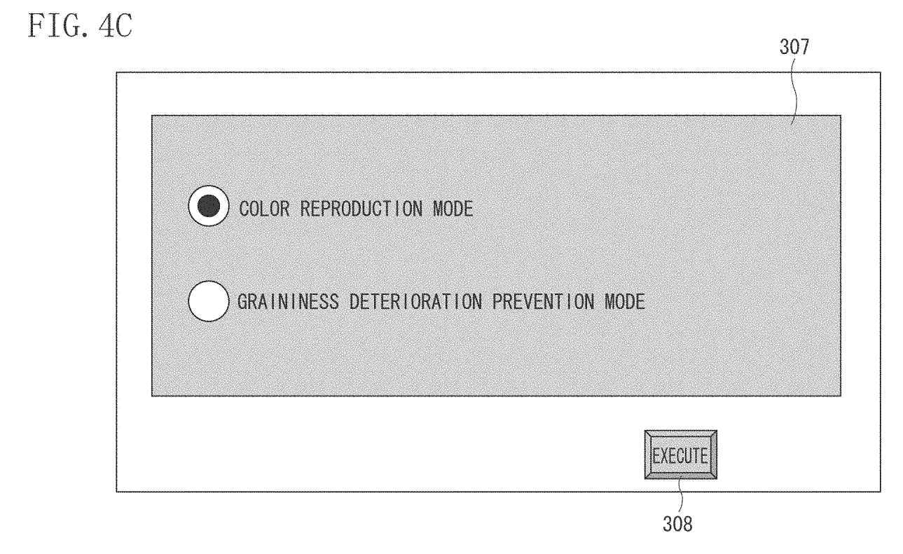

First, in step S1, the display control unit 201 displays on the monitor 109 the UI screen configured to receive information for the execution of image processing from the user. FIG. 4A illustrates an example of the UI screen. An instruction input area 301 is a region for inputting an instruction regarding color information (color information 1 and color information 2) specifying colors to be reproduced in two different azimuthal directions. An instruction input area 302 is a region for inputting an instruction regarding the numbers of vertical and horizontal pixels of an image to be output. An output button 303 is a button for executing a process of generating formation data and outputting the generated data to the image forming unit 108. An end button 304 is a button for executing an end operation and closing the UI screen. If a user instruction via the instruction input areas 301 and 302 is received and an instruction to press the output button 303 is given, the processing proceeds to step S2.

In step S2, the acquisition unit 202 acquires the color information 1 and the color information 2 as the color information which is input as the user instruction and specifies the colors to be reproduced in the two different azimuthal directions. The color information 1 and the color information 2 specify two different colors. In the present exemplary embodiment, the color information is International Commission on Illumination (CIE) tristimulus values XYZ. The format of the color information is not limited to the CIE tristimulus values XYZ and can be any other format such as red-green-blue (RGB) values, CIEL*a*b* (CIELAB) values, or CIE color appearance model (CIECAM) 02 values.

In step S3, the determination unit 203 determines a combination of recording amounts of a plurality of types of colored inks to be used to form an anisotropic print that reproduces the acquired color information 1 and the acquired color information 2. Details of the processing executed in step S3 will be described below. In step S4, the generation unit 204 generates formation data based on the combination of recording amounts which is determined in step S3. Details of the processing executed in step S4 will be described below. In step S5, the output unit 205 outputs the formation data generated in step S4 to the image forming unit 108, and the process is ended.

### <Combination of Recording Amounts of Colored Inks>

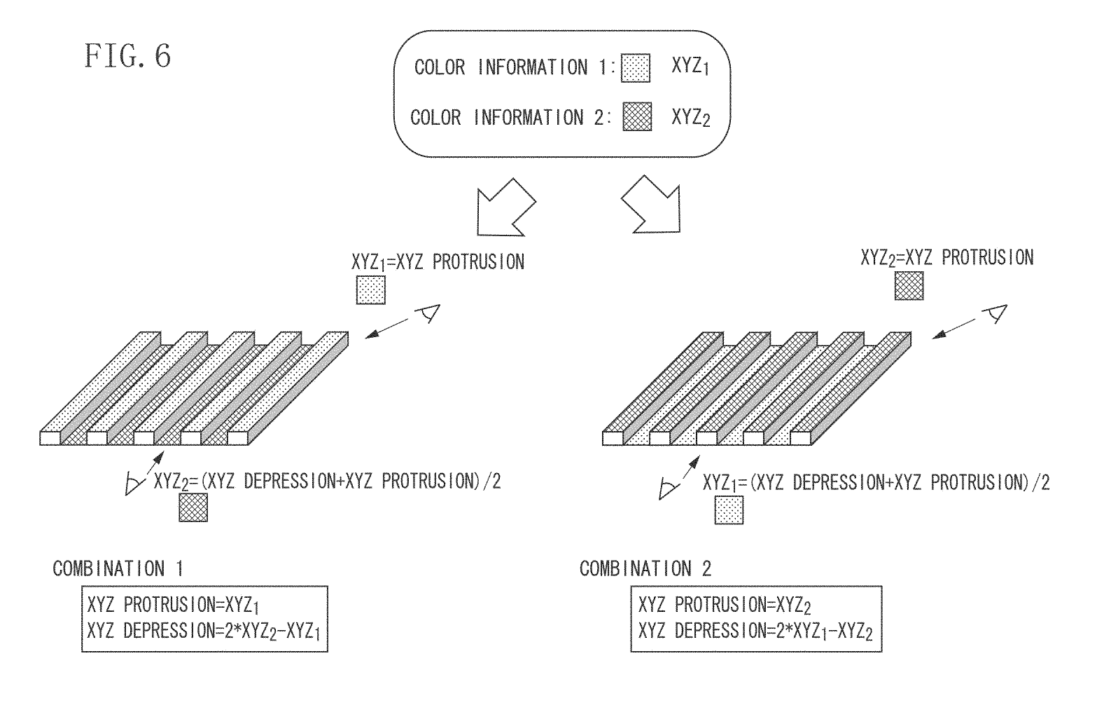

The following describes the process of determining a combination of recording amounts of colored inks in step S3. First, prior to the description of the flow of the process, the combination of recording amounts of colored inks for forming an anisotropic print described above will be described with reference to FIG. 6. As illustrated in FIG. 6, there are two combinations of recording amounts of colored inks for reproducing the color (XYZ₁) specified by the color information 1 and the color (XYZ₂) specified by the color information 2 in two directions of different azimuth angles. For example, in order to reproduce XYZ₁ in the viewpoint 1 and XYZ₂ in the viewpoint 2, XYZ₁ is to be expressed by the color (XYZ protrusion) of the colored inks to be recorded on the protruded portion. Further, XYZ₂ is to be expressed by a mixed color ((XYZ protrusion+XYZ depression)/2) of the colors of the colored inks to be recorded on the protruded portion and the depressed portion. Thus, in the case where the protruded portion and the depressed portion have the same area, a combination of recording amounts of colored inks is determined such that the colors of the colored inks to be recorded on the protruded and depressed portions satisfy the XYZ protrusion and the XYZ depression calculated by formula (1) below.

XYZ protrusion=XYZ₁,XYZ depression=2XYZ₂−XYZ₁ formula (1).

Alternatively, the colors XYZ₂ and XYZ₁ can be reproduced in the viewpoints 1 and 2, respectively. In this case, the color XYZ₂ is to be expressed by the colors (XYZ protrusion) of the colored inks to be recorded on the protruded portion, and the color XYZ₁ is to be expressed by the mixed color ((XYZ protrusion+XYZ depression)/2) of the colors of the colored inks to be recorded on the protruded portion and the depressed portion. Thus, in the case where the protruded portion and the depressed portion have the same area, a combination of recording amounts of colored inks is determined such that the colors of the colored inks to be recorded on the protruded and depressed portions satisfy the XYZ protrusion and the XYZ depression calculated by formula (2) below.

XYZ protrusion=XYZ₂,XYZ depression=2XYZ₁−XYZ₂ formula (2).

As described above, unless one of the colors specified respectively by the color information 1 and the color information 2 is determined as the color to be reproduced in one of two directions of different azimuth angles and the other color is determined as the color to be reproduced in the other direction, there exist two combinations of recording amounts of colored inks. In the present exemplary embodiment, one of the above-described two combinations that has a smaller total recording amount of the colored inks to be recorded on the protruded portion is selected so that the colored inks recorded on the protruded portion are less likely to flow into the depressed portion. Thus, in the process according to the present exemplary embodiment, which will be described in detail below, the same combination of recording amounts is recorded even if the colors specified respectively by the color information 1 and the color information 2 instructed on the UI screen illustrated in FIG. 4A are replaced by each other.

As described above, in the present exemplary embodiment, in the case of reproducing the color information 1 and the color information 2 in different azimuth angles, one of the color information 1 and the color information 2 is expressed by the color of the colored inks on the protruded portion while the other one is expressed by additive color mixing of the colored inks on the protruded and depressed portions. Since one of the colors is expressed by additive color mixing, there is a limitation on the combination (color information 1 and color information 2) of colors that can be expressed in different azimuth angles. For example, a combination of colors which are same in hue and different in brightness or saturation, e.g., red and pink, can be reproduced using inks which are different in brightness or saturation and are the same in hue such that the protruded portion is red and the depressed portion is white or the protruded portion is pink and the depressed portion is dark red. Further, a combination of similar colors which are different in hue, such as red and orange, can be reproduced using a combination of inks having hues which are changed by additive color mixing such that the protruded portion is red and the depressed portion is yellow or the protruded portion is orange and the depressed portion is red-violet. However, for example, a combination of complementary colors, such as red and green, and a combination of colors which are significantly different in brightness, such as white and black, cannot be reproduced because additive color mixing of any color to red or white will not produce green or black.

### <Process of Determining Combination of Recording Amounts of Colored Inks (Step S3)>

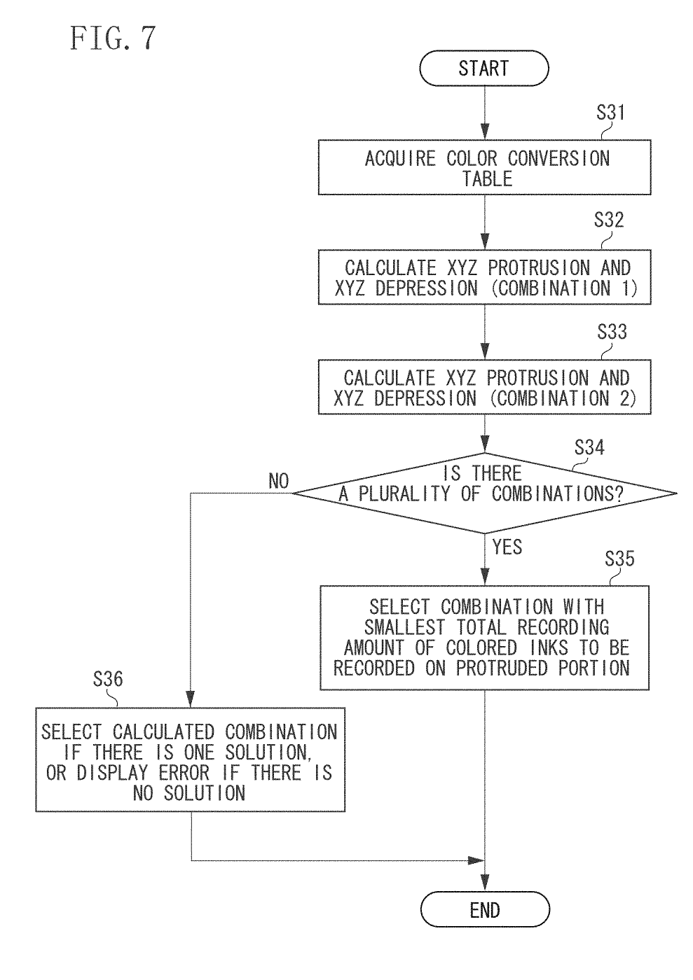

A flow of the process of determining a combination of recording amounts of colored inks (step S3) will be described with reference to FIG. 7. FIG. 7 is a flowchart illustrating the process executed in step S3.

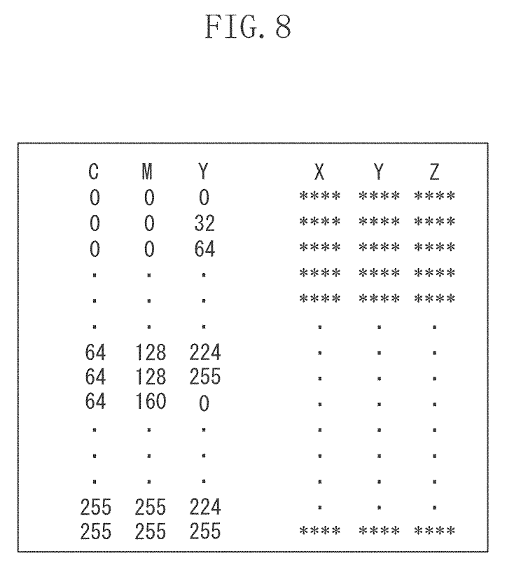

In step S31, a color conversion table of the image forming unit 108 is acquired. As illustrated in FIG. 8, the color conversion table in the present exemplary embodiment specifies colors (CIE tristimulus values XYZ) to be reproduced according to the recording amounts of colored inks C (cyan), M (magenta), and Y (yellow) employed by the image forming unit 108. Specifically, the color conversion table is data in which the recording amounts CMY of the colored inks are associated with the CIE tristimulus values XYZ. The color conversion table is generated in advance by forming patches on a recording medium with the colored inks in different recording amounts and then measuring the colors of the formed patches. Then, the color conversion table is stored in a storage device such as the HDD 103.

In step S32, a combination of recording amounts of colored inks for use in reproducing the color specified by the color information 1 (XYZ₁) in the viewpoint 1 and the color specified by the color information 2 (XYZ₂) in the viewpoint 2 is determined. Specifically, first, the XYZ protrusion and the XYZ depression are calculated using formula (1) described above. A combination of recording amounts of colored inks (CMY protrusion and CMY depression) is acquired from the color conversion table acquired in step S31 using the calculated XYZ protrusion and the calculated XYZ depression. The acquisition is conducted by a reverse lookup of the color conversion table using publicly-known interpolation processing such as cubic interpolation or tetrahedral interpolation. If there is no combination of recording amounts of colored inks for reproducing the color specified by the color information 1 (XYZ₁) in the viewpoint 1 and the color specified by the color information 2 (XYZ₂) in the viewpoint 2, it is determined that there is no solution.

In step S33, a combination of recording amounts of colored inks for reproducing the color specified by the color information 2 (XYZ₂) in the viewpoint 1 and the color specified by the color information 1 (XYZ₁) in the viewpoint 2 is determined. Specifically, first, the XYZ protrusion and the XYZ depression are calculated using formula (2) described above. A combination of recording amounts of colored inks (CMY protrusion and CMY depression) is acquired from the color conversion table acquired in step S31 using the calculated XYZ protrusion and the calculated XYZ depression. The acquisition is conducted by a reverse lookup of the color conversion table using publicly-known interpolation processing such as cubic interpolation or tetrahedral interpolation. If there is no combination of recording amounts of colored inks for reproducing the color specified by the color information 2 (XYZ₂) in the viewpoint 1 and the color specified by the color information 1 (XYZ₁) in the viewpoint 2, it is determined that there is no solution.

In step S34, whether there is a plurality of combinations of recording amounts of colored inks (CMY protrusion and CMY depression) is determined. If there is a plurality of combinations (YES in step S34), the processing proceeds to step S35. On the other hand, if a plurality of combinations (NO in step S34) does not exist, the processing proceeds to step S36.

In step S35, a more suitable combination of recording amounts is selected from the CMY protrusion and the CMY depression acquired in step S32 and the CMY protrusion and the CMY depression acquired in step S33. Specifically, the CMY protrusion with the smallest total recording amount of the recording amounts of colored inks to be recorded on the protruded portion is selected to prevent the colored inks recorded on the protruded portion from flowing into the depressed portion. If the selected CMY protrusion is the CMY protrusion acquired in step S32, the CMY depression acquired in step S33 is selected for the CMY depression. On the other hand, if the selected CMY protrusion is the CMY protrusion acquired in step S33, the CMY depression acquired in step S32 is selected for the CMY depression. As described above, a combination of recording amounts of colored inks to be recorded on the depressed portion is determined based on the determined combination of recording amounts of colored inks to be recorded on the protruded portion so that the protruded portion and the depressed portion express different colors. In the present exemplary embodiment, the total recording amount is the total amount obtained by adding the recording amounts of C, M, and Y together. By the above-described processing, the colored inks to be recorded on the protruded portion become less likely to flow into the depressed portion, so a decrease in the difference between the colors to be reproduced in the two different directions is prevented, and variations of how the colors are observed according to the observation angle are visually recognizable with ease.

Step S36 is the processing which is executed in the case where there is one or no combination of recording amounts. In the case where the number of acquired combinations of recording amounts is one, the acquired combination is selected. In the case where there is no combination of recording amounts, an error is displayed on the monitor 109 via the UI screen, and the process is ended.

### <Process of Generating Formation Data (Step S4)>

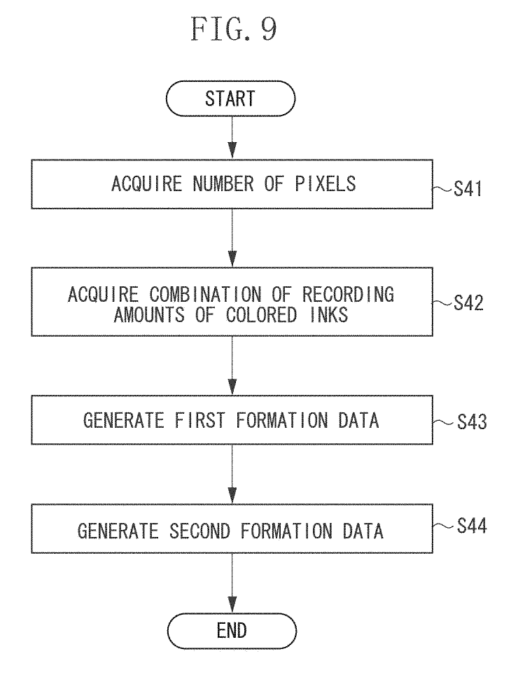

FIG. 9 is a flowchart illustrating the process executed in step S4.

In step S41, the number of horizontal pixels and the number of vertical pixels in an image to be output which are input and instructed by the user via the instruction input area 302 are acquired. In the description of the subsequent processing in the present exemplary embodiment, the number of vertical pixels is 200, and the number of horizontal pixels is 200. In step S42, the combination of recording amounts of colored inks (CMY protrusion and CMY depression) determined in step S3 is acquired.

In step S43, first formation data for forming the uneven shape (uneven layer) corresponding to the number of horizontal pixels and the number of vertical pixels acquired in step S41 is generated. In the present exemplary embodiment, the height and cycle of the protrusions and depressions are determined in advance, and the height is 110 μm and the cycle is 10 pixels (corresponding to approximately 106 μm at 1200 dpi for each of the depressed portion and the protruded portion) in the description of the subsequent processing. The first formation data is data in which the recording amounts of inks (UV inks) for forming the protrusions and depressions and the number of times of layering for the image forming unit 108 to form the protrusions and depressions are recorded in each pixel. If the height of the protruded portion obtained when the image forming unit 108 records the UV inks by 100% is 20 μm, the pixels corresponding to the protruded portion are recorded six times including five times of recording by 100% and one time of recording by 50%. Further, the height of the depressed portion is 0 μm, so the pixels corresponding to the depressed portion do not need the UV ink recording. Thus, the recording amounts for the region corresponding to the protruded portion and the region corresponding to the depressed portion are set to 550% and 0%, respectively, and they are switched every five pixels. This is repeated 20 cycles to generate formation data. The height and cycle set in the present exemplary embodiment are mere examples and can be set freely according to the print resolution and observation angle (elevation angle direction) in actual implementation.

In step S44, second formation data for forming a colored layer with the colored inks on the uneven layer is generated. Specifically, second formation data is generated in which the CMY protrusions are recorded on the pixels corresponding to the protruded portion and the CMY depressions on the pixels corresponding to the depressed portion based on the combination of recording amounts of colored inks (CMY protrusion and CMY depression) acquired in step S42. Then, the process is ended. The second formation data is not limited to the data specifying the recording amounts of colored inks. For example, the second formation data can be binary data for each recording scan in which results of publicly-known halftone processing and path decomposition executed using the recording amounts of colored inks are recorded.

As described above, the combination of colored inks which has the smallest total recording amount is selected to reduce the recording amounts of colored inks to be recorded on the protruded portion at the time of forming the printed matter with the surface having the uneven shape. In this way, the inks recorded on the protruded portion become less likely to flow into the depressed portion, so desired colors are reproduced with great accuracy. Further, the colored inks are recorded on the uneven shape such that the colors are observed differently when observed from different directions, so color anisotropy is reproduced on the recording medium.

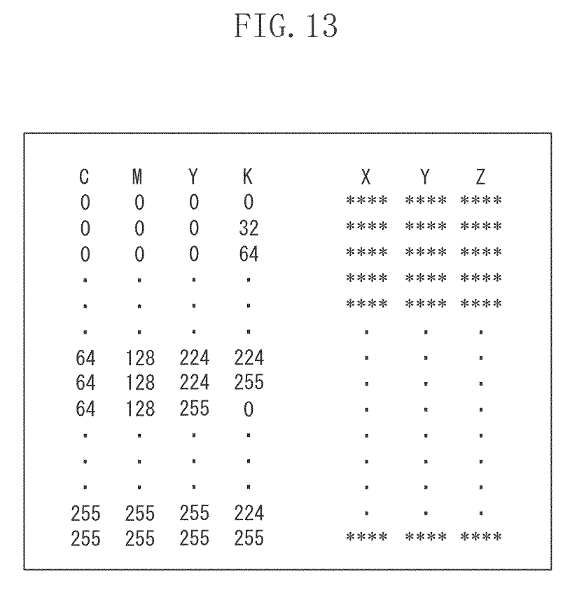

In the present exemplary embodiment, the colored inks employed by the image forming unit 108 are the inks of three colors of CMY, so there are one combination of recording amounts of colored inks to be recorded on the protruded portion and one combination of recording amounts of colored inks to be recorded on the depressed portion, but the disclosure is not limited to the above-described example. For example, the image forming unit 108 can employ a K (black) ink in addition to the CMY inks. In this case, there exist a plurality of combinations of recording amounts of colored inks to be recorded on the protruded portion and a plurality of combinations of recording amounts of colored inks to be recorded on the depressed portion due to under color removal (UCR). Thus, in the color conversion table acquisition in step S31, a table is acquired in which a plurality of CMYK combinations is associated with one XYZ as illustrated in FIG. 13. In step S32, a plurality of CMYK protrusions corresponding to the calculated XYZ protrusion is acquired, and a plurality of CMYK depressions corresponding to the calculated XYZ depression is acquired. Step S33 is similar to step S32. Then, in step S35, the CMYK protrusion that has the smallest total recording amount is selected from the plurality of CMYK protrusions acquired in steps S32 and S33. If the selected CMYK protrusion is a CMYK protrusion acquired in step S32, one of the plurality of CMYK depressions acquired in step S33 is selected as the CMYK depression. On the other hand, if the selected CMYK protrusion is a CMYK protrusion acquired in step S33, one of the plurality of CMYK depressions acquired in step S32 is selected as the CMYK depression. At the time of selecting one CMYK depression from the plurality of CMYK depressions, one of the plurality of CMYK depressions that has the greatest total recording amount is selected to prevent a deterioration of graininess due to UCR processing. As described above, the present exemplary embodiment is applicable to the cases in which the inks employed by the image forming unit 108 are not the inks of three colors of CMY.

In the first exemplary embodiment, the method in which the combination having the smallest total recording amount of the colored inks to be recorded on the protruded portion is selected to reduce the recording amounts of colored inks to be recorded on the protruded portions of the uneven shape is described. In a second exemplary embodiment, a method of determining a combination of recording amounts of colored inks based on the viscosities of the colored inks to be recorded on the protruded portion will be described. The hardware and logical configurations of the image processing apparatus 1 in present exemplary embodiment are similar to those in the first exemplary embodiment. A difference from the first exemplary embodiment is the process of determining a combination of recording amounts of colored inks in step S3. Thus, the process executed in step S3 will be described in detail below.

### <Process of Determining Combination of Recording Amounts of Colored Inks (Step S3)>

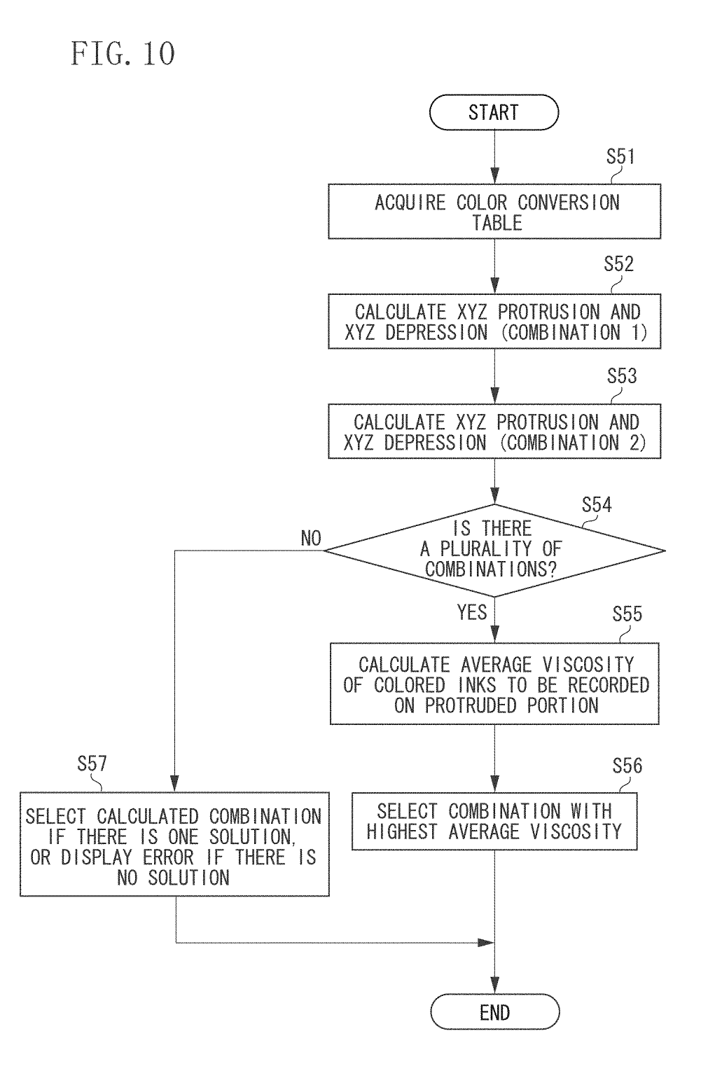

FIG. 10 is a flowchart illustrating the process executed in step S3.

In step S51, the color conversion table of the image forming unit 108 is acquired. In the present exemplary embodiment, the color conversion table is data in which the recording amounts of colored inks CMY are associated with the CIE tristimulus values XYZ and the viscosities of the colored inks. The viscosities of the colored inks are the viscosities of the respective colored inks (C, M, Y) employed by the image forming unit 108. In general, data which specifies the viscosity of a colored ink is provided by the manufacturer of the colored ink. Further, viscosimeters are commercially available, so the values are obtainable with ease.

The processing executed in step S52 is similar to the processing executed in step S32 in the first exemplary embodiment, so description of step S52 is omitted. The processing executed in step S53 is similar to the processing executed in step S33 in the first exemplary embodiment, so description of step S53 is omitted. In step S54, whether there is a plurality of combinations of recording amounts of colored inks (CMY protrusion and CMY depression) is determined. If there is a plurality of combinations (YES in step S54), the processing proceeds to step S55. On the other hand, if a plurality of combinations (NO in step S54) does not exist, the processing proceeds to step S56.

In step S55, step S52, the average viscosity of the colored inks to be recorded on the protruded portion is calculated for each of the combinations of recording amounts of colored inks which are calculated in steps S52 and S53. An average viscosity V is calculated using formula (3) below, where Cᵥ, Mᵥ, and Yᵥ are the viscosities of the respective colored inks, and Cₐ, Mₐ, and Yₐ are the recording amounts of the respective colored inks. In formula (3), the average value of the viscosities weighted according to the recording amounts of colored inks is calculated (weighted average).

$$V = \frac{CvCa + MvMa + YvYa}{Ca + Ma + Ya} \qquad Formula ( 3 )$$

In step S56, one combination with the highest average viscosity of the colored inks to be recorded on the protruded portion is selected from the plurality of combinations of recording amounts of colored inks by reference to the average viscosity calculated in step S55. If the selected CMY protrusion is a CMY protrusion acquired in step S52, the CMY depression acquired in step S53 is selected as the CMY depression. On the other hand, if the selected CMY protrusion is a CMY protrusion acquired in step S53, the CMY depression acquired in step S52 is selected as the CMY depression. Step S57 is the processing which is executed in the case where there is one or no combination of recording amounts. In the case where the number of acquired combinations of recording amounts of colored inks is one, the acquired combination is selected. In the case where there is no combination of recording amounts, an error is displayed on the monitor 109 via the UI screen, and the process is ended.

As described above, the combination of colored inks with the highest average viscosity of the colored inks to be recorded on the protruded portion is selected at the time of forming the printed matter with the surface having the uneven shape. In this way, the inks recorded on the protruded portion become less likely to flow into the depressed portion, so desired colors are reproduced with great accuracy. Further, the colored inks are recorded on the uneven shape such that the colors are observed differently when observed from different directions, whereby color anisotropy is reproduced on the recording medium.

While the example in which the colored inks employed by the image forming unit 108 are the inks of three colors of CMY is described in the present exemplary embodiment, the colored inks employed by the image forming unit 108 can be inks of four colors of CMYK as in the first exemplary embodiment.

While the combination of inks with the highest average viscosity is selected in the present exemplary embodiment, a combination with the highest minimum viscosity can be selected.

In the above-described exemplary embodiments, the example in which the direction in which the color specified by the color information 1 is to be reproduced and the direction in which the color specified by the color information 2 is to be reproduced out of the two directions having different azimuth angles are not determined is described. In a third exemplary embodiment, an example in which the color specified by the color information 1 (XYZ₁) is designated in advance as the color to be reproduced in the viewpoint 1 and the color specified by the color information 2 (XYZ₂) is designated in advance as the color to be reproduced in the viewpoint 2 will be described. Specifically, in the present exemplary embodiment, the color information 1 specifies the color to be reproduced in the case of observing the printed matter from the viewpoint specified in FIG. 5A, and the color information 2 specifies the color to be reproduced in the case of observing the printed matter from the viewpoint 2. The image forming unit 108 in the present exemplary embodiment employs colored inks of four colors of CMYK. The hardware and logical configurations of the image processing apparatus 1 in the present exemplary embodiment are similar to those in the first exemplary embodiment. A difference from the first exemplary embodiment is the process of determining a combination of recording amounts of colored inks in step S3. Thus, the process executed in step S3 will be described in detail below.

### <Process of Determining Combination of Recording Amounts of Colored Inks (Step S3)>

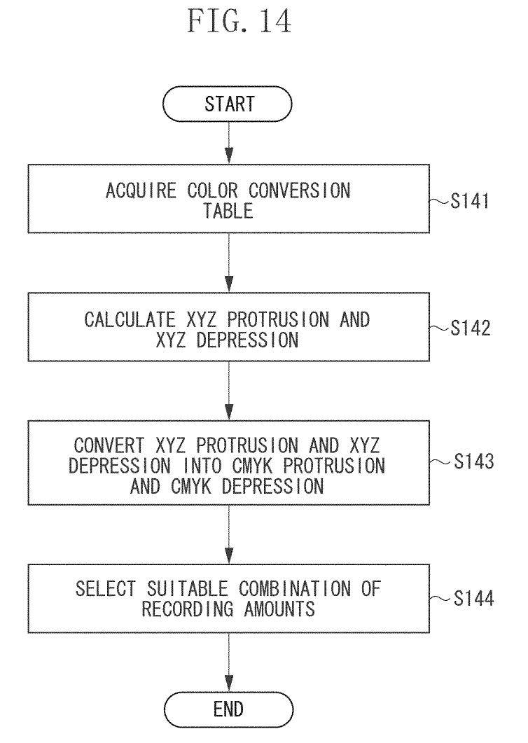

The following describes the process of determining a combination of recording amounts of colored inks in step S3. A flow of the process executed in step S3 will be described below with reference to FIG. 14. FIG. 14 is a flowchart illustrating the process executed in step S3.

In step S141, the color conversion table of the image forming unit 108 is acquired. As illustrated in FIG. 13, the color conversion table in the present exemplary embodiment specifies the colors (CIE tristimulus values XYZ) to be reproduced according to the recording amounts of colored inks C (cyan), M (magenta), Y (yellow), and K (black) employed by the image forming unit 108. Specifically, the color conversion table is data in which the recording amounts CMYK of the colored inks are associated with the CIE tristimulus values XYZ. The color conversion table is generated in advance by forming patches on a recording medium with the colored inks in different recording amounts and then measuring the colors of the formed patches. Then, the color conversion table is stored in a storage device such as the HDD 103.

In step S142, the color (XYZ protrusion) of the colored inks to be recorded on the protruded portion and the color (XYZ depression) of the colored inks to be recorded on the depressed portion are calculated based on the color information 1 and the color information 2 acquired in step S2. In order to reproduce the color specified by the color information 1 (XYZ₁) in the viewpoint 1 and the color specified by the color information 2 (XYZ₂) in the viewpoint 2 as in a combination in FIG. 6, XYZ₁ is to be expressed by the color (XYZ protrusion) of the colored inks to be recorded on the protruded portion. Further, XYZ₂ is to be expressed by a mixed color ((XYZ protrusion+XYZ depression)/2) of the colors of the colored inks to be recorded on the protruded portion and the depressed portion. Thus, in the case where the protruded portion and the depressed portion have the same area, the XYZ protrusion and the XYZ depression are calculated by formula (1) described above.

In step S143, the recording amounts (CMYK protrusion and CMYK depression) of colored inks are acquired based on the XYZ protrusion and the XYZ depression calculated in step S142. The acquisition is conducted by a reverse lookup of the color conversion table illustrated in FIG. 13 using publicly-known interpolation processing such as cubic interpolation or tetrahedral interpolation. The color conversion table herein specifies the CIE tristimulus values XYZ with respect to the recording amounts CMYK of colored inks and is obtained by executing publicly-known UCR processing on the colored inks CMY. Thus, there is a plurality of CMYK combinations with respect to XYZ. From all the acquired CMYK candidates with respect to XYZ, a suitable combination is selected in step S144.

In step S144, a suitable combination of recording amounts CMYK is selected from the CMYK candidates acquired in step S143. Specifically, as to the protruded portion, the CMYK protrusion with the smallest total recording amount of the recording amounts of colored inks to be recorded on the protruded portion is selected to prevent the colored inks recorded on the protruded portion from flowing into the depressed portion. As to the depressed portion, the CMYK depression with the greatest total recording amount of the recording amounts of colored inks to be recorded on the depressed portion is selected to prevent a deterioration of graininess. In the present exemplary embodiment, the total recording amount is the total amount obtained by adding the recording amounts of C, M, Y, and K together. By the above-described processing, the colored inks recorded on the protruded portion become less likely to flow into the depressed portion, so a decrease in the difference between the colors to be reproduced in the two different directions is prevented, and variations of how the colors are observed according to the observation angle are visually recognizable with ease.

As described above, in the printer which employs the four colors of CMYK, a plurality of combinations of recording amounts of colored inks is calculated by UCR processing. In the present exemplary embodiment, the combination of colored inks which has the smallest total recording amount is selected to reduce the recording amounts of colored inks to be recorded on the protruded portion at the time of forming the printed matter with the surface having the uneven shape using the printer. In this way, the inks recorded on the protruded portion become less likely to flow into the depressed portion, so desired colors are reproduced with great accuracy. Further, the colored inks are recorded on the uneven shape such that the colors are observed differently when observed from different directions, so color anisotropy is reproduced on the recording medium.

In the first and third exemplary embodiments, the method is described in which the combination with the smallest total recording amount of colored inks to be recorded on the protruded portion is selected to reduce the recording amounts of colored inks to be recorded on the protruded portion of the uneven shape. In a fourth exemplary embodiment, a method will be described in which a maximum allowable total recording amount is set with respect to the recording amounts of colored inks to be recorded on the protruded portion and a combination of recording amounts with the total recording amount not exceeding the set maximum allowable total recording amount is selected. The hardware and logical configurations of the image processing apparatus 1 in the present exemplary embodiment are similar to those in the first exemplary embodiment. A difference from the first exemplary embodiment is the process of determining a combination of recording amounts of colored inks in step S3. Thus, the process executed in step S3 will be described in detail below.

### <Process of Determining Combination of Recording Amounts of Colored Inks (Step S3)>

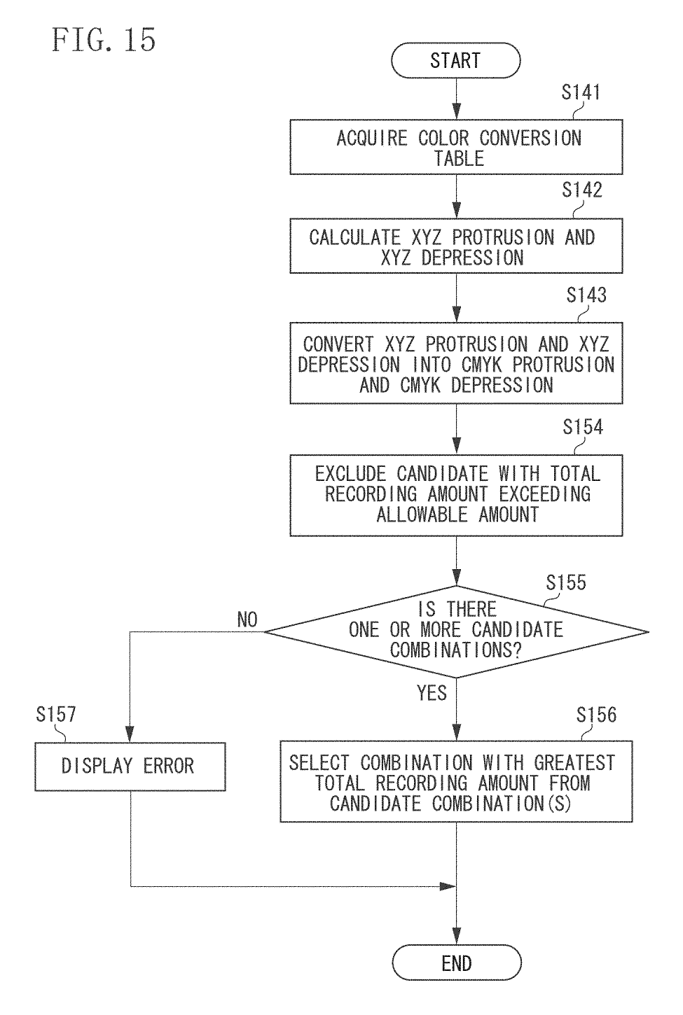

FIG. 15 is a flowchart illustrating the process executed in step S3.

Steps S141, S142, and S143 are similar to those in the third exemplary embodiment, so description thereof is omitted.

In step S154, whether the total recording amount of the colored inks to be recorded on the protruded portion exceeds the maximum allowable total recording amount (hereinafter, “allowable amount” or “threshold value”) is determined for each combination of recording amounts of colored inks acquired in step S143. The allowable amount is a value which is preset based on ink properties such as viscosity. If the total recording amount of a combination exceeds the allowable amount, the colored inks recorded on the protruded portion are likely to flow into the depressed portion, so the combination is excluded from the candidates.

In step S155, whether there is one or more candidate combinations of recording amounts of colored inks to be recorded on the protruded portion is determined. If there is no candidate (NO in step S155), the total recording amount of the colored inks to be recorded on the protruded portion is not adjustable to the allowable amount or less, so in step S157, an error is output to display the error on the monitor 109. On the other hand, if there is one or more candidates (YES in step S155), the total recording amount of the colored inks to be recorded on the protruded portion is adjustable to the allowable amount or less, so in step S156, a suitable combination of recording amounts is selected.

In step S156, a suitable recording amount is selected from the candidate combinations of recording amounts of colored inks to be recorded on the protruded portion. Specifically, a combination with the greatest total recording amount is selected as CMYK protrusion from the candidates with the total recording amount not exceeding the allowable amount. As to the depressed portion, the CMYK depression with the greatest total recording amount of the recording amounts of colored inks to be recorded on the depressed portion is selected to prevent a deterioration of graininess.

Step S157 is the processing which is executed in the case where the total recording amount of the colored inks to be recorded on the protruded portion is not adjustable to the allowable amount or less. In this case, it is not possible to print the user-input color information 1 and color information 2 with the colored inks having the total recording amount not exceeding the allowable amount, so an error is displayed on the monitor 109 via the UI screen, and the process is ended.

As described above, the combination of recording amounts of colored inks to be recorded on the protruded portion which has the greatest total recording amount among the combinations with the total recording amount not exceeding the threshold value is selected at the time of forming the printed matter with the surface having the uneven shape. In this way, the colored inks recorded on the protruded portion become less likely to flow into the depressed portion while a deterioration of graininess due to UCR processing is prevented, so desired colors are reproduced with great accuracy. Further, the colored inks are recorded on the uneven shape such that the colors are observed differently when observed from different directions, so color anisotropy is reproduced on the recording medium.

While the example in which the error is displayed on the monitor 109 via the UI screen in step S157 of the present exemplary embodiment is described above, the present exemplary embodiment is not limited to the above-described example. In step S157, a UI screen which prompts the user to give an instruction regarding whether to switch the color information 1 and the color information 2 and execute the process of step S3 again can be displayed. If a user instruction to switch the color information 1 and the color information 2 and execute the process of step S3 again is input, the color information 1 and the color information 2 are switched and the process of step S3 is executed again. On the other hand, if a user instruction not to switch the color information 1 and the color information 2 and not to execute the process of step S3 again is input, the process is ended.

While the methods of selecting a combination with the smallest total recording amount of the colored inks to be recorded on the protruded portion, etc. are described in the above-described exemplary embodiments, the method is not limited to the above-described methods. For example, according to a first modified example, a combination of recording amounts can be determined taking into consideration the graininess and the allowable amount of the recording amounts of colored inks. In this case, patches are formed on a recording medium with the colored inks in different recording amounts, and the graininess of each of the patches is evaluated in advance.

While the four types of colored inks of C, M, Y, and K are described as the color inks employed by the image forming unit 108 in the third and fourth exemplary embodiments, the colored inks are not limited to the above-described example. In a second modified example, low-density inks such as light cyan (Lc) and light magenta (Lm) can be used in addition to C, M, Y, and K. In this case, color density decomposition is executed to replace Lc by C, Lm by M, etc., increasing the combinations of recording amounts of colored inks. From the combinations, for example, a combination of recording amounts of colored inks with the smallest total recording amount can be selected. Further, besides the low-density inks described above, spot color inks such as red (R), green (G), and blue (B) inks can be used.

While the case in which the protruded portion and the depressed portion of the protrusions and depressions formed on the recording medium have the same area is described as an example in the above-described exemplary embodiments, the areas of the protruded portion and the depressed portion can be different. In this case, for example, in a third modified example, when the area ratio between the protruded portion and the depressed portion is n:1, the XYZ protrusion and the XYZ depression are calculated using formula (4) obtained by generalizing formula (1).

XYZ protrusion=XYZ₁XYZ depression=(n+1)XYZ₂−nXYZ₁ formula(4).

Further, formula (2) can also be generalized in this way.

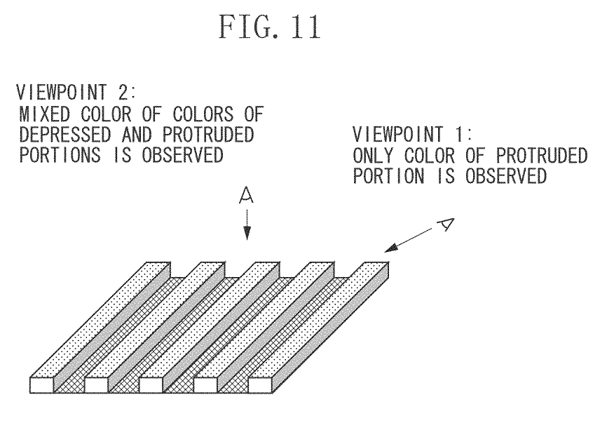

While the colors to be reproduced when the printed matter is observed from the two viewpoints of different azimuth angles are input is described in the above-described exemplary embodiments, the exemplary embodiments are not limited to the example. In a fourth modified example, as illustrated in FIG. 11, since the printed matter having a surface with the uneven shape is observed differently from different directions of elevation angles of the viewpoints, the colors to be reproduced when the printed matter is observed from two viewpoints of different elevation angles can be input as well.

While the example in which an instruction regarding the two pieces of color information is input by directly inputting numerical values using the UI screen illustrated in FIG. 4A is described in the above-described exemplary embodiments, the two pieces of color information can be input using image data. For example, in a fifth modified example, two pieces of image data are input as first image data and second image data, and color information recorded on each pixel of the first image data is determined as color information 1 whereas color information recorded on each pixel of the second image data is determined as color information 2. Further, one piece of 6-channel image data in which the color information 1 and the color information 2 are recorded on each pixel can be input. Alternatively, image data in which the color information 1 is recorded on each pixel can be input, and a numerical value input by the user using the UI screen can be used as the color information 2. In the case of inputting the image data, the processing of the above-described exemplary embodiments is applied to each pixel, and a printed matter having anisotropy different in each position (region) is formed.

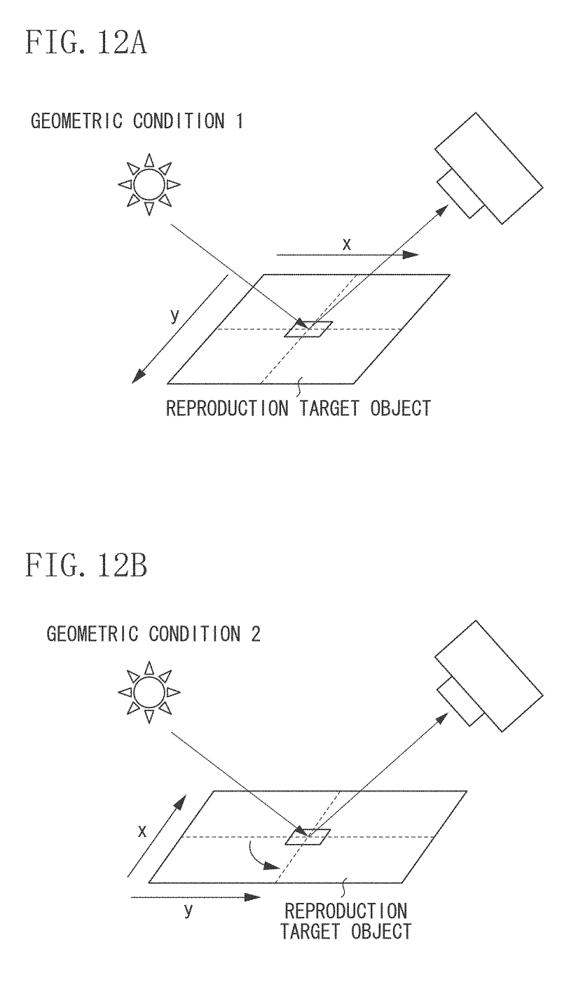

In the case of inputting the image data, the image data is generated in advance and stored in a storage device such as the HDD 103. As to the image data, for example, as illustrated in FIG. 12, two pieces of data which are image data acquired by capturing an image of a reproduction target object under a geometric condition 1 and image data acquired by capturing an image of a reproduction target object under a geometric condition 2 can be used. Differences due to the geometric condition are corrected by publicly-known distortion correction processing such as affine transformation. Further, the image data is not limited to the captured images and can be image data generated according to a user instruction input via the UI screen.

While the colored inks are used as colored recording materials in the above-described exemplary embodiments, the colored recording materials are not limited to the above-described example. For example, in a sixth modified example, colored toners can be used as the colored recording materials. Further, while the UV curable resin inks (UV inks) are used as inks for forming protrusions and depressions, inks curable with light other than ultraviolet rays can be used.

While the example in which the first formation data and the second formation data are generated is described in the above-described exemplary embodiments, the disclosure is not limited to the above-described example. For example, in a seventh modified example 7, first formation data generated in advance and stored in a storage device such as the HDD 103 can be acquired to generate second formation data based on the first formation data and the determined combination of recording amounts of colored inks. Further, information specifying the determined combination of recording amounts of colored inks can be output directly without generating the first formation data and the second formation data.

While a combination of recording amounts of colored inks is determined based on the total recording amount of the colored inks to be recorded on the protruded portion in the first, third, and fourth exemplary embodiments or based on the average viscosity of the colored inks to be recorded on the protruded portion in the second exemplary embodiment, the determination is not limited to the above-described example. For example, in an eighth modified example, a combination can be determined based on both the total recording amount and the average viscosity. For example, a weighted average of evaluation values of the total recording amount and the average viscosity can be taken to select a combination of recording amounts of colored inks with a high evaluation. Further, a combination of recording amounts of colored inks which are less likely to flow can be determined based on ink properties other than the total recording amount and viscosity. For example, the surface tensions of the colored inks are applicable.

While the example in which one piece of color information 1 and one piece of color information 2 are input and the colors specified by the color information 1 and the color information 2 are applied to an entire image to be formed is described in the above-described exemplary embodiments, the disclosure is not limited to the above-described example. The two pieces of color information can be changed for each image region. For example, in a ninth modified example, the color information 1 and the color information 2 in a first region of an image respectively specify red and green, and the color information 1 and the color information 2 in a second region, which is different from the first region, of the image respectively specify blue and yellow. As to a region separation method, for example, the regions can be separated based on a user instruction via the UI screen. Alternatively, a method can be used in which image data is input and objects in images specified by the image data are detected to separate regions by borders between the objects.

While the example in which the uneven shape is applied to an entire image is described in the above-described exemplary embodiments, the uneven shape can be formed only on a part of image. In this case, for example, in a tenth modified example, binary data indicating a region where the uneven shape is to be formed and the regions where the uneven shape is not to be formed is generated based on a user instruction via the UI screen. The recording amounts of UV inks of a region of the first formation data corresponding to the region specified as the region where the uneven shape is not to be formed according to the binary data is set to 0%. As to a region of the second formation data corresponding to the region specified as the region where the uneven shape is not to be formed according to the binary data, the second formation data is generated such that the recording amounts of one of the CMY protrusion and the CMY depression, which is the determined combination of recording amounts, are recorded on each pixel.

While the example in which the uneven shape with the pattern of parallel lines is formed is described in the above-described exemplary embodiments, the uneven shape is not limited to the above-described example. The uneven shape can be any uneven shape as long as a printed matter is formed such that the colors are observed differently when the observation angle is changed in the azimuthal direction. For example, in an eleventh modified example, an uneven shape including a plurality of protruded portions having a bottom surface with an aspect ratio which differs for each region of the image is formed.

In the first and third exemplary embodiments, the combination of recording amounts of colored inks to be recorded on the protruded portion with the smallest total recording amount is selected. In the second exemplary embodiment, the combination of recording amounts of colored inks to be recorded on the protruded portion with the highest average viscosity is selected. In the fourth exemplary embodiment, the combination of recording amounts of colored inks to be recorded on the protruded portion with the greatest total recording amount is selected among the candidates with the total recording amount not exceeding the allowable amount. The above-described examples can be used in combination. For example, in a twelfth modified example, a combination which is optimum from a particular standpoint, such as a standpoint that the total recording amount is the smallest amount or a standpoint that the average viscosity is the highest average viscosity, is displayed on the UI screen in the step of selecting one of a plurality of combinations of recording amounts. Then, a user instruction regarding the standpoint from which an optimum combination will be selected as the combination of recording amounts of colored inks to be recorded on the protruded portion is received via the UI screen illustrated in FIG. 4B. Specifically, the user is prompted to select on an instruction input area 305 a standpoint from which an optimum combination is to be selected as a combination of recording amounts of colored inks to be recorded on the protruded portion. Thereafter, if an instruction to press an execution button 306 is input, a combination of recording amounts of colored inks to be recorded on the protruded portion is selected based on the instruction input in the instruction input area 305. The UI is not limited to the UI illustrated in FIG. 4B and, for example, a UI as illustrated in FIG. 4C can be displayed to enable the user to perform intuitive input. In this case, the user is prompted to designate in an instruction input area 307 one of a color reproduction mode and a graininess deterioration prevention mode. Thereafter, if an instruction to press an execution button 308 is input, a combination of recording amounts of colored inks to be recorded on the protruded portion is selected based on the instruction received in the instruction input area 307. The color reproduction mode is a mode in which an optimum combination is selected from the standpoint that the total recording amount is the smallest or the average viscosity is the highest. The graininess deterioration prevention mode is a mode in which an optimum combination is selected from the standpoint that the total recording amount is the greatest among the candidates with the total recording amount not exceeding the allowable amount.

Further, a user instruction as to the priority (priority order) of the above-described standpoints can be received in advance via the UI screen in step S1 besides the color information and the number of pixels. In this case, an optimum combination is searched for from the standpoint of the highest priority according to the priority order, and if there is no optimum combination, then an optimum combination is searched for from the standpoint of the second highest priority.

The example in which the color information (color information 1 and color information 2) specifying the colors to be reproduced in the two different azimuthal directions is input and the XYZ protrusion and the XYZ depression are calculated from the color information 1 and the color information 2 is described in the above-described exemplary embodiments. However, the disclosure is not limited to the above-described example. For example, in a thirteenth modified example, the XYZ protrusion and the XYZ depression can be input directly without the use of the color information 1 and the color information 2. In this case, as in the case of inputting the color information 1 and the color information 2, input of a user instruction regarding the XYZ protrusion and the XYZ depression is received via the UI screen as illustrated in FIG. 4A. The two colors to be input here are two different colors, and it is unnecessary to determine one of the colors as the color to be recorded on the protruded portion and the other as the color to be recorded on the depressed portion. In this case, one of the two different colors is the XYZ protrusion and the other one is the XYZ depression, so there are at least two combinations of recording amounts of colored inks. In a case where the input two colors are the same color, an error may be displayed on the monitor 109.

While one of the plurality of combinations of recording amounts of colored inks to be recorded on the protruded portion is selected in the above-described exemplary embodiments, the process is not limited to the above-described example. For example, in a fourteenth modified example, whether to execute the combination selection described above can be determined according to a user instruction. Specifically, a UI is displayed to prompt the user to designate a mode in which the combination selection is executed or a mode in which one predetermined combination is used. Whether to apply the processing according to the above-described exemplary embodiments is determined according to a user instruction input via the UI. Further, the processing can be as follows. One combination of recording amounts of colored inks is held in advance for a piece of color information, and whether the acquired combination of recording amounts corresponding to the color information is allowable from the standpoint of the total recording amount and the viscosity is determined. If it is determined that the acquired combination is not allowable, the plurality of acquired combinations with respect to the color information is calculated, and one of the plurality of combinations is selected. On the other hand, if it is determined that the acquired combination is allowable, a combination held in advance is determined as a combination of recording amounts of colored ink.

### Other Embodiments

Embodiment(s) of the disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)), a flash memory device, a memory card, and the like.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2017-079152, filed Apr. 12, 2017, and No. 2017-243381, filed Dec. 20, 2017, which are hereby incorporated by reference herein in their entirety.

What is claimed is:

1. An apparatus configured to determine a first recording amount of a colored recording material to be recorded on a protruded portion and a second recording amount of a colored recording material to be recorded on a depressed portion to form an image by recording plurality of types of colored recording materials on the protrusion and the depression on the recording medium so that different colors are visually recognized from the image when the image is observed from at least two different directions of azimuth angles, the apparatus comprising:
a first acquisition unit configured to acquire color information specifying a first color and a second color;
a calculation unit configured to calculate based on the color information:
a first color combination of a color to be recorded on the protruded portion and a color to be recorded on the depressed portion for a case in which the first color is visually recognized when the image is observed from a first direction and the second color is visually recognized when the image is observed from a second direction, and
a second color combination of the color to be recorded on the protruded portion and the color to be recorded on the depressed portion for a case in which the second color is visually recognized when the image is observed from the first direction and the first color is visually recognized when the image is observed from the second direction;
a second acquisition unit configured to acquire a first recording amount combination of the first recording amount and the second recording amount based on the first color combination and acquire a second recording amount combination of the first recording amount and the second recording amount based on the second color combination; and
a determination unit configured to select one combination according to a predetermined condition from the first recording amount combination and the second recording amount combination.

2. The apparatus according to claim 1, wherein the first color specified by the color information or the second color specified by the color information is a color expressed by a mixed color of the color to be recorded on the protruded portion and the color to be recorded on the depressed portion.

3. The apparatus according to claim 1, wherein the determination unit determines the first recording amount and the second recording amount by selecting from the first recording amount combination and the second recording amount combination a combination by which the colored recording material recorded on the protruded portion does not flow into the depressed portion.

4. The apparatus according to claim 3, wherein the determination unit determines the first recording amount and the second recording amount by selecting from the first recording amount combination and the second recording amount combination a combination with a smallest total recording amount of the colored recording material to be recorded on the protruded portion.

5. The apparatus according to claim 3, wherein the determination unit determines the first recording amount and the second recording amount by selecting from the first recording amount combination and the second recording amount combination one combination by which a total recording amount of the colored recording material to be recorded on the protruded portion does not exceed a predetermined threshold value.

6. The apparatus according to claim 5, wherein the determination unit determines the first recording amount and the second recording amount by selecting a combination with a greatest total recording amount of the colored recording material to be recorded on the protruded portion from a combination with a total recording amount not exceeding a predetermined threshold value out of the first recording amount combination and the second recording amount combination.

7. The apparatus according to claim 3, wherein the determination unit determines the first recording amount and the second recording amount by selecting from the first recording amount combination and the second recording amount combination a combination with a highest average viscosity of the colored recording material to be recorded on the protruded portion.

8. The apparatus according to claim 7, wherein the average viscosity is a weighted average value of viscosities of the plurality of types of colored recording materials to be recorded on the protruded portion which is weighted according to the recording amount of each of the colored recording materials.

9. The apparatus according to claim 3, wherein the determination unit determines the first recording amount and the second recording amount by selecting from the first recording amount combination and the second recording amount combination a combination with a highest minimum viscosity of the colored recording material to be recorded on the protruded portion.

10. The apparatus according to claim 3, wherein the determination unit determines based on a user instruction the first recording amount and the second recording amount by selecting one combination from three combinations which are:
a combination with a smallest total recording amount of the colored recording material to be recorded on the protruded portion from the first recording amount combination and the second recording amount combination,
a combination with a greatest total recording amount of the colored recording material to be recorded on the protruded portion among a combination with a total recording amount of the colored recording material to be recorded on the protruded portion which does not exceed a predetermined threshold value from the first recording amount combination and the second recording amount combination, and
a combination with a highest average viscosity of the colored recording material to be recorded on the protruded portion from the first recording amount combination and the second recording amount combination.

11. The apparatus according to claim 1, wherein the first recording amount combination is a plurality of combinations of the first recording amount and the second recording amount, and the second recording amount combination is a plurality of combinations of the first recording amount and the second recording amount.

12. The apparatus according to claim 1, wherein the determination unit determines the first recording amount and the second recording amount by selecting from the first recording amount combination and the second recording amount combination a combination with a greatest total recording amount of the colored recording material to be recorded on the depressed portion.

13. The apparatus according to claim 1, wherein the first acquisition unit acquires the color information input via a user interface.

14. The apparatus according to claim 1, wherein a difference between an azimuth angle of the first direction and an azimuth angle of the second direction is 90 degrees.

15. The apparatus according to claim 1, wherein, in a case where an instruction to determine the first recording amount such that the colored recording material recorded on the protruded portion does not flow into the depressed portion is received, the determination unit determines the first recording amount and the second recording amount by selecting one combination according to a predetermined condition from the first recording amount combination and the second recording amount combination, and
wherein in a case where no instruction to determine the first recording amount such that the colored recording material recorded on the protruded portion does not flow into the depressed portion is received, the determination unit determines a combination of predetermined recording amounts according to the color information as a combination of the first recording amount and the second recording amount.

16. The apparatus according to claim 1, further comprising:
a third acquisition unit configured to acquire first formation data for forming the protrusion and the depression on the recording medium; and
a generation unit configured to generate second formation data for forming a colored layer by recording the colored recording material on the protrusion and the depression based on the first recording amount and the second recording amount.

17. The apparatus according to claim 16, further comprising a forming unit configured to form the protrusion and the depression on the recording medium and form the colored layer on the formed depression and the formed protrusion based on the first formation data and the second formation data.

18. The apparatus according to claim 17, wherein the forming unit forms the protrusion and the depression using an ink which is curable by being irradiated with light, and the forming unit forms the colored layer using a colored ink as the colored recording material.

19. method of determining a first recording amount of a colored recording material to be recorded on a protruded portion and a second recording amount of a colored recording material to be recorded on a depressed portion to form an image by recording the plurality of types of colored recording materials on the protrusion and the depression on the recording medium so that different colors are visually recognized from the image when the image is observed from at least two different directions of azimuth angles, the method comprising:
acquiring color information specifying a first color and a second color;
calculating, based on the color information, a first color combination of a color to be recorded on the protruded portion and a color to be recorded on the depressed portion for a case in which the first color is visually recognized when the image is observed from a first direction of the two directions and the second color is visually recognized when the image is observed from a second direction;
calculating, based on the color information, a second color combination of the color to be recorded on the protruded portion and the color to be recorded on the depressed portion for a case in which the second color is visually recognized when the image is observed from the first direction and the first color is visually recognized when the image is observed from the second direction;
acquiring a first recording amount combination of the first recording amount and the second recording amount based on the first color combination;
acquiring a second recording amount combination of the first recording amount and the second recording amount based on the second color combination; and
selecting one combination according to a predetermined condition from the first recording amount combination and the second recording amount combination.

20. A non-transitory computer-readable storage medium storing instructions that, when executed by a computer, cause the computer to perform a method of determining a first recording amount of a colored recording material to be recorded on a protruded portion and a second recording amount of a colored recording material to be recorded on a depressed portion to form an image by recording the plurality of types of colored recording materials on the protrusion and the depression on the recording medium so that different colors are visually recognized from the image when the image is observed from at least two different directions of azimuth angles which are different from each other, the image processing method comprising:
acquiring color information specifying a first color and a second color;
calculating, based on the color information, a first color combination of a color to be recorded on the protruded portion and a color to be recorded on the depressed portion for a case in which the first color is visually recognized when the image is observed from a first direction of the two directions and the second color is visually recognized when the image is observed from a second direction;
calculating, based on the color information, a second color combination of the color to be recorded on the protruded portion and the color to be recorded on the depressed portion for a case in which the second color is visually recognized when the image is observed from the first direction and the first color is visually recognized when the image is observed from the second direction;
acquiring a first recording amount combination of the first recording amount and the second recording amount based on the first color combination;
acquiring a second recording amount combination of the first recording amount and the second recording amount based on the second color combination; and
selecting one combination according to a predetermined condition from the first recording amount combination and the second recording amount combination.
